(19) United States
(12) Patent Application Publication
(10) Pub. No.: US 2024/0066183 A1
(43) Pub. Date: Feb. 29, 2024

(54) Amniotic Membrane Product and Packaging

(71) Applicant: Luicina Patent Holdco, LLC, Loveland, CO (US)

(72) Inventor: Ryan Dregalla, Windsor, CO (US)

(21) Appl. No.: 17/896,604

(22) Filed: Aug. 26, 2022

(51) Int. Cl. A61L 27/36; A61L 27/54

(52) U.S. Cl. CPC A61L 27/3604; A61L 27/54

(57) ABSTRACT

A wet human or other tissue product may be packaged with a primary and secondary backing. The primary backing may cover a larger area of the tissue than the secondary backing, and the primary backing may be removed first during application of the tissue to a patient, either by sliding or peeling the primary backing away. The secondary backing may assist in holding the tissue in place while the primary backing is removed. The primary backing may provide mechanical support and keep the tissue spread during packaging, handling, and application to a patient. The tissue assembly with primary and secondary backing may be stored in a heat sealed, sterile flat pouch with preservation media. The packaged assembly may include a port for introducing preservation media. A use case for the packaging includes wet, undried amniotic membrane.

### BACKGROUND

Human placentas have been found to have many useful tissues and fluids for various therapies and treatments. Among those tissues is the amniotic membrane.

Amniotic membrane consists of amnion and chorion, with an intermediate layer in between. The intermediate layer is sometimes called the spongy layer, and in various texts, may be associated with the amnion, while in other texts, the spongy layer may be associated with the chorion.

Amniotic membranes have been used in various forms in surgery for wound treatment. Sometimes, the amniotic membranes are lyophilized and injected into joints, wounds, or other uses. In other cases, membranes may be dried then later rehydrated when placed in a wound.

### SUMMARY

A wet human or other tissue product may be packaged with a primary and secondary backing. The primary backing may cover a larger area of the tissue than the secondary backing, and the primary backing may be removed first during application of the tissue to a patient, either by sliding or peeling the primary backing away. The secondary backing may assist in holding the tissue in place while the primary backing is removed. The primary backing may provide mechanical support and keep the tissue spread during packaging, handling, and application to a patient. The tissue assembly with primary and secondary backing may be stored in a heat sealed, sterile flat pouch with preservation media. The packaged assembly may include a port for introducing preservation media. A use case for the packaging includes wet, undried amniotic membrane.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

In the drawings,

FIG. 1 is a pictorial illustration of an embodiment showing a packaged membrane tissue.

FIG. 2 is a pictorial illustration of an embodiment showing a sequence of applying a membrane using a primary and a secondary backing.

FIG. 3 is a pictorial illustration of an embodiment showing a sequence of applying a membrane using a second type of tissue packaging assembly.

FIG. 4 is a diagram illustration of a first tissue packaging assembly showing a primary backing, a secondary backing, and a graft tissue.

FIG. 5 is a diagram illustration of a second tissue packaging assembly showing a primary backing, a secondary backing, and a graft tissue.

FIG. 6 is a diagram illustration of a third tissue packaging assembly showing a primary backing, a secondary backing, and a graft tissue.

FIG. 7 is a diagram illustration of a fourth tissue packaging assembly showing a primary backing, a secondary backing, and a graft tissue.

FIG. 8 is a diagram illustration of a fifth tissue packaging assembly showing a primary backing, a secondary backing, and a graft tissue.

FIG. 9 is a diagram illustration of a sixth tissue packaging assembly showing a primary backing, a secondary backing, and a graft tissue.

FIG. 10 is a diagram illustration of a seventh tissue packaging assembly showing a primary backing, a secondary backing, and a graft tissue.

### DETAILED DESCRIPTION

Packaging for Wet Tissue Used in Surgery

Many tissues derived from human, bovine, porcine, or other sources may be used in human or veterinary surgery. These tissues may be difficult to handle and store when wet, especially for thin membranes such as amniotic membranes. Because of this difficulty, there are many tissue products that are dried, morselized, or have other secondary processes.

Very thin membranes may be particularly difficult to handle as the membrane may bunch up, stretch, tear, or fold over on itself. Many tissue products may consist of several layers of membrane, and the flatwise orientation of the membrane may be desired during surgery, where one side of the membrane is preferentially placed against the patient.

Wet tissues are dissected from a donor and stored without drying, freezing, and with minimal processing. For example, amniotic membrane may be harvested from a placenta donor for the various tissues in the placenta. One such tissue may be an amniotic graft that may consist of the amniotic epithelium, basement membrane, stromal layer, and, sometimes, the fibroblast layer.

In some cases, the full thickness of amniotic membrane may be used. Such a membrane may include the various layers of the amnion and chorion.

Such an amniotic graft may be a section of tissue comprising several layers and having a preferred orientation for surgery. Typically, such a graft may be positioned with the stromal layer towards the patient. In such tissue products, a primary membrane may be placed on the opposite side of the tissue such that the stromal layer is exposed and the patient.

Wet tissue membranes may be packaged by placing the tissue on a primary backing such that the side facing the patient is exposed. A secondary backing may be used to assist in removing the primary backing. When used in surgery, the assembly may be placed against the patient, then the primary backing removed, and the secondary backing may then be removed. After removing the backings, the tissue may be in place with the proper flatwise orientation.

Many multi-layered tissues may be difficult to identify which side is which. Many such tissues may be translucent, partially translucent, or have similar color for both sides of the tissue, even though one side of the tissue may be “up” and the other “down” with respect to the patient.

By orienting the tissue against a backing such that the preferred patient-facing side is exposed, a surgeon may not have to perform an inspection to find the proper flat-wise orientation. Such flat-wise orientation may be performed in a laboratory environment when the tissue may be harvested and prepared for storage.

The assembly of the backings and the tissue may have enough mechanical strength and stiffness for a surgeon to remove the assembly from its packaging, observe and inspect the tissue, and place the assembly on the patient. The backings may be a thin polymer sheet, mesh, or other material that may be bendable and flexible, but generally stiffer than the tissue.

The assembly may be stored in a flat pouch with preservation media for transportation and storage. In many cases, an appropriate preservation and storage media may allow a tissue to remain surgically useful in room temperature storage for several days, months, or even years. Some tissues may be stored in low temperature storage above or below freezing, although many wet tissues may change their therapeutic characteristics if frozen.

Amniotic Membrane for Surgery and Other Therapies

Amniotic membrane may be packaged and stored wet for use in surgeries and other therapies. When the membrane is packaged, stored, and used without drying, the membrane may retain many of its inherent constituents, such as lubricin, hyaluronic acid, and other constituents.

Drying amniotic membrane causes many of the chemical and protein constituents to degrade. In many cases, drying causes many constituents to lose all or much of their effectiveness.

Dried amniotic membrane has an advantage that it is easy to manipulate. A dried membrane may typically be a rigid, thin piece of translucent material that is placed on a wound, then rehydrated.

In contrast, wet amniotic membrane may be difficult to manipulate and place into position during surgery. When picking up a wet, somewhat sticky translucent membrane, it is easy for the membrane to fold over on itself or otherwise become bunched up.

A surgeon handling such a membrane may then have to gently tease the membrane into position by pulling the edges of the membrane into position. This careful, tedious manipulation can be time consuming in an expensive operating room environment or during emergent situations.

Many membranes in their wet state, including amniotic membranes, have a small amount of tensile strength and will tolerate being pulled gently without tearing or ripping. Further, the membrane in its wet state often has a good amount of elasticity. This means that the membrane can stretch when pulled. The elasticity may allow a surgeon to manipulate the membrane to cover a wound bed, for example, that is deep or highly curved.

Membrane that has been dried and subsequently rehydrated typically does not have as much elasticity. Many dried membrane products are crosslinked, either intentionally or through various processes, so to give the dried product much more strength.

The product of an amniotic membrane that is stored and used in its wet state has been shown to be much more effective in various treatments than dried amniotic membranes. One of the benefits of wet, never dried membrane may be the much higher elasticity of the membrane.

Amniotic membrane may be various sized portions of intact membrane. During harvesting, the membrane may be separated from the amniotic sac, then portions of the membrane may be cut to size. Each portion may be separated and stored in individual packaging for use.

In many cases, the portions of intact membrane may be chosen from sections of the membrane that are not torn, ripped, cut, or otherwise damaged. Such portions that may be torn or damaged may be separated and, for example, used for products that are lyophilized.

The membrane may have an orientation notch or other feature that may indicate which side of the membrane is facing upwards. One such version may be a vertical notch that may be cut in the upper right hand corner of a membrane, such that if the membrane were placed incorrectly, the vertical notch would be positioned incorrectly.

Packaging Wet Membrane

A wet membrane may be packaged between two backing materials. A primary backing material may have a relatively large pore structure and be mechanically stronger than a secondary backing material with a smaller pore structure and a weaker mechanical structure.

The primary backing material may have enough mechanical strength to transfer and manipulate the sandwich of the two backing materials and the membrane section. In a typical use case, the sandwiched assembly may be removed from a pouch or other packaging, then held with forceps and placed in position.

The primary backing material may be selected to be rigid enough to support the sandwiched assembly during removal from packaging and placement on a patient. The mechanical strength may be sufficient so that the assembly or the membrane does not fold over on itself during handling. In many cases, the primary backing material may be somewhat flexible so that the assembly may be placed on a patient and manipulated to conform to the shape of the area being treated.

The secondary backing material may have similar or less rigidity than the primary backing material. Further, the secondary backing material may be less porous or have smaller pores than the primary backing material.

The difference in pore size between the primary and secondary backing material may allow the primary backing material to slide off the membrane with less friction force, stickiness, or drag than the secondary backing material.

In one example, the primary backing material may have a pore size of 5 micron in a polypropylene sheet, while the secondary backing material may be a polyethersulfone sheet having a 0.45 micron pore size.

The primary and secondary backing material may have differences in texture. The primary backing material, for example, may have a smoother texture to allow the primary backing material to slide gently off the membrane. In such an example, the secondary backing material may have a more coarse or rough texture to help hold the membrane in place during the sliding operation. Such an effect may be obtained by different pore sizes between the backing, as well as through embossed or other texturing applied to the backing materials.

Further, the physical and visual differences between the primary and secondary backing materials may indicate the side of the amniotic membrane. For example, the primary backing material may be a colorless translucent material, while the secondary backing material may be opaque and colored. Other examples may include a primary backing material that may be tinted one color while the secondary backing material may be tinted a second color.

The primary backing may be at least somewhat hydrophilic. In such situations, the membrane may be attracted to the primary backing and resist falling away from the primary backing when the assembly may be manipulated during assembly or when a surgeon places the assembly on a patient.

In other configurations, the primary backing may be at least somewhat hydrophobic. In such situations, the primary backing may be more easily slid or peeled away from the membrane when placing the membrane on a patient. Even though the primary backing may be at least slightly hydrophobic, the wet consistency of the tissue may hold the tissue against the primary backing during assembly and manipulation.

Some configurations may use a hydrophilic material that may have pores. One such version may be a mesh material. The hydrophilic material may attract the tissue to the primary backing, but the pores may limit that amount of attraction, thereby allowing the primary backing to be slid or peeled away. Such a tradeoff may allow a packaging designer to balance the ‘stickiness’ of the tissue to the primary backing with the ease by which the primary backing may be removed during surgery.

In the same way, the primary backing and the secondary backing may be formed of different materials. The different materials may be simple color differences, which may help a surgeon visually identify the primary backing material from the secondary backing material. In many cases, the primary backing material may be translucent to allow a surgeon to inspect the tissue prior to applying to the patient.

In some cases, the primary backing material and secondary backing material may have different mechanical characteristics, such as stiffness, hydrophilic/hydrophobic, pore size, and others. The differences in materials may ease the method of removing the primary backing material during surgery first, then removing the secondary backing material.

Amniotic Membrane

The amniotic membrane contains several different layers, which may include the epithelium, basement membrane, compact stromal layer, and the fibroblast layer. Some embodiments may have removed the fibroblast layer, thereby exposing the compact or stromal layer.

Many treatments have the stomal layer against the patient and the epithelium layer outwards. The stromal layer can be stickier than the epithelium layer and adhere to the patient during placement. The physical and visual differences between the backing materials may be chosen so that a physician can determine verify that the stromal layer is, indeed, being placed against the patient, for example.

However, the differences between the stickiness of the epithelium layer and the stromal layer may be imperceptible to a surgeon. By making structural and visual differences between the primary and secondary backing materials, the surgeon may not need to investigate the orientation of the membrane.

In some cases, printing, embossing, or other features may be incorporated into one of the primary or secondary backing materials. The printing, embossing, or other features may verify the orientation of the membrane. In one such use case, the primary or secondary backing material may be printed with the words “UP”, “DOWN”, or other text to visually indicate orientation during application.

Method of Preparation

The membrane may be dissected from a placenta, then prepared for packaging. The membrane may be washed with saline as well as physically scraped to remove intermediate or spongy layer.

The membrane may be decellularized using several different methods, such as mechanical scraping, washing, chemical or enzymatic decellularization, or other methods. In many cases, the membrane may be washed using a decellularization media.

The membrane may be removed from the decellularizing media and spread on a flat surface. A DNA stain may be performed to determine the orientation of epithelium or stromal side, and the stromal side may be placed down. In some products, the epithelium layer may be placed down. Such orientation may be noted on the packaging and product for selection by a surgeon.

The membrane may be cut into desired shapes, and each cut piece may be placed on the primary pre-cut backing material. The secondary backing material may be placed on top of the membrane to form a sandwich assembly.

In some cases, the membrane may be spread across a large piece of primary backing material, then the membrane and primary backing material may be cut simultaneously into smaller assemblies.

The sandwich assembly may be placed in a heat-sealable pouch, and preservation medial may be added. The pouch may then be heat sealed.

Some packaging may include a syringe port that may be heat sealed into the packaging. The syringe port may be used to inject a predefined amount of preservation media, then to extract any air bubbles from the packaging.

The packaging may be a pouch or envelope having at least one clear side. Such a pouch may be heat sealed. Some packaging may have a peel-away feature that allows the package to be opened by peeling apart two layers of the packaging. Other packaging may be opened by cutting the packaging using surgical scissors or a scalpel.

A clear side of the packaging may allow visual inspection to see the membrane as it is placed on the primary backing material, as well as to inspect any air bubbles in the packaging. In general, air in the packaging may oxidize or degrade the membrane, so as much air may be removed from the packaging as possible prior to sealing. In packages with a syringe or other port, the port may be used after heat sealing to mechanically remove any remaining air. In some packaging systems, a small amount of air may be left in the package.

The package may be placed inside a secondary pouch or packaging. The primary package containing the membrane and backing material sandwich may be sterilized and placed into a secondary, external package. Such an arrangement may allow the secondary, external package to be sterilized before bringing into an operating theater or other sterile environment, then opened within the sterile environment to remove the internal package.

Applying Wet Membrane

The wet membrane may be applied to a mammal for treatment by placing the sandwich assembly on the area for treatment, then removing the primary backing material, holding the membrane in place, then removing the secondary backing from the membrane.

The membrane may be removed from packaging and held with forceps. The mechanical strength of the sandwich assembly may be sufficient to keep the membrane from folding over on itself or bunching up.

The sandwich assembly may be placed on the wound or treatment area with the tissue touching the patient. The primary backing material may be gently slid off or peeled from the membrane, leaving the membrane in place.

The pore size, hydrophilic nature, or other mechanical properties of the primary backing may decrease the adhesion of the membrane to the primary backing so that the membrane may remain in place during removal of the primary backing. The mechanical properties of the primary backing may be selected such that the tissue may adhere or cling to the primary backing when the sandwich may be handled by a surgeon, but the mechanical properties may be balanced against the ease by which the primary backing may be removed from the tissue.

Once the primary backing may be removed, the surgeon may hold the membrane in place, then gently slide or peel the secondary backing away, thereby causing the membrane to come in contact with the area for treatment. In some cases, the membrane may be sutured in place, at least in part, prior to removing the secondary backing.

The primary backing may extent to the full surface or planar area of the tissue. In some cases, the primary backing may extend past the tissue in one or more dimensions. In other cases, the primary backing may be sized to be close to or even smaller than the tissue on one or more dimensions. In many cases, the primary backing may cover at least 75%, 90%, or more of the surface area of the tissue.

The primary backing may have additional features in its shape, such as a finger notch, pull tab, peel tab, or other feature. A finger notch may be useful to hold the tissue in place while the primary backing is being removed.

The primary backing may be removed by sliding or pulling the primary backing off the membrane tissue. In such a process, the primary backing may be slid in the same plane as the tissue, with the wet preservation media providing a shear or slip plane in which the items may be separated. In such a use case, a pull tab may extend past the planar area of the tissue, allowing the surgeon to grasp the pull tab with forceps. Without the pull tab, the surgeon may separate the primary backing from the tissue to grasp the primary backing.

The primary backing may be removed by peeling or lifting the primary backing. In such a process, the primary backing may be lifted on one side or corner and peeled back, leaving the tissue in place. In such a use case, a peel tab may be placed past the planar area of the tissue, allowing the surgeon to grasp the peel tab with forceps. In many cases, a peel operation may be performed by teasing apart the primary backing from the tissue, then lifting the primary backing away.

The secondary backing may not extend across the full planar or surface area of the membrane. The secondary backing may cover less than 10%, 25%, 50%, or 75% of the planar area of the membrane tissue. The secondary backing may serve to hold the tissue in place while the primary backing may be removed.

The secondary backing may extend outward, away from the tissue such that at least a portion of the secondary backing may not be in contact with the tissue. In many cases, the secondary backing may extend outward, away from at least a portion of the primary backing material as well. The secondary backing may provide an area for a surgeon to hold the tissue in place with a finger, for example, while the primary backing may be removed.

In some use cases, the secondary backing may be between the tissue and the patient. In such a use case, the secondary backing may adhere to the tissue and help hold the tissue in place while the primary backing may be removed. The secondary backing may adhere to the tissue through greater affinity because of pore size, material selection, or other mechanisms. In this use case, the secondary backing may hold the tissue in position while the primary backing may be slid off or peeled away from the tissue.

In other uses cases, the secondary backing may be placed between the primary backing and the tissue. In such a use case, the tissue may be placed on the patient and the full surface area of the tissue may be positioned against the patient. The secondary backing may then be held in place while the primary backing may be removed by sliding or peeling.

Once the primary backing may be removed, the secondary backing may then be removed. The secondary backing may have a pull tab or other feature that extends past the edge of the membrane so that a surgeon may be able to easily grasp the tab during the removal process.

Preservation Media

Preservation media may be a liquid media in which a tissue may be stored. In some cases, the preservation media may be derived from plasma or other fluid captured from a donor. In other cases, the preservation media may be saline or other non-derived liquid.

Preservation media may include additives, such as nutrients, drugs, or other materials that may allow the tissue to remain in a usable state. In some cases, such additives may provide therapeutic benefits, such as expediting healing, reducing tissue rejection, or other benefits. In some cases, the preservation media may contain anti-microbial agents or other materials that may preserve the tissue.

In the case of amniotic tissue, the preservation media may be derived from amniotic or other human fluids, including umbilical cord fluids. In many cases, human amniotic fluid may be filtered, centrifuged, and concentrated to form a preservation media.

Sterilization Process

The tissue and backing assembly may be assembled using sterile materials, such as sterile tissue and sterile backing materials. After assembling the tissue and backing materials, the assembly may be placed in a heat sealed pouch, which, again, may be sterile at the time of assembly.

The heat sealed pouch may have preservation media added. In many cases, a port may be heat sealed into the pouch to allow preservation media to be added and for air to be removed. The pouch may then be placed in a second, outer envelope, which may also be heat sealed.

The envelope and pouch assembly may be irradiated or otherwise sterilized. Once sterilized, the tissue assembly may be stored at room temperature or cooler until used.

Some assemblies may be manufactured in a sterile environment and samples may be tested over time to determine that the product remains sterile.

Throughout this specification, like reference numbers signify the same elements throughout the description of the figures.

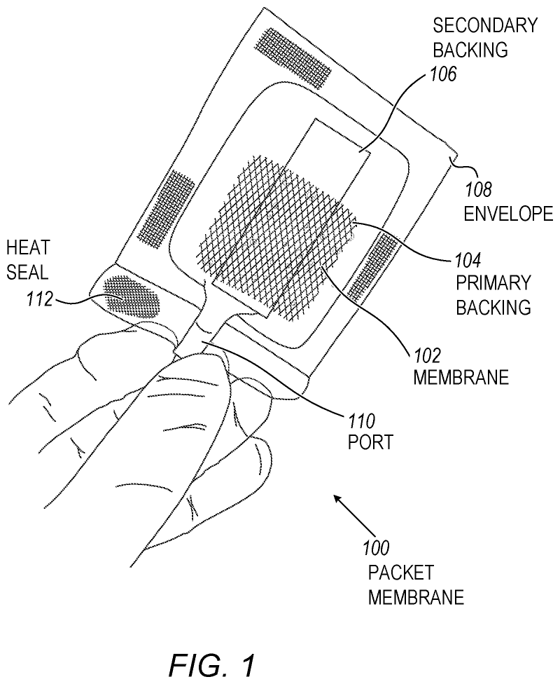

FIG. 1 is an illustration of an example 100 of a packaged membrane. The membrane 102 may be sandwiched between a primary backing 104 and a secondary backing 106.

The primary backing 104 may cover most or all of the surface area of the membrane 102. In some cases, the primary backing 104 may be larger than the surface area of the membrane 102 and may extend past the edge of the membrane 102 by several millimeters or more on one or more edges. In some cases, the membrane 102 may extend past an edge of the primary backing 104.

In general, the primary backing 104 may be selected to help keep the membrane fully extended during storage and application to the patient. A typical amniotic membrane may be very soft and pliable, and in many cases, the membrane 102 may become bunched up and folded. Once in such a state, the membrane 102 may be difficult to spread onto a patient.

The primary backing 104 may be more stiff and less pliable than the membrane 102. The primary membrane 104 may hold the membrane 102 from folding over on itself or becoming bunched.

The membrane 102 may be wet membrane that may be harvested, decellularized, cut to size, and otherwise prepared. The wet membrane 102 may adhere to the primary backing 104 by fluid surface tension. In such a state, the wet membrane 102 may ‘stick’ to the primary backing 104, yet the primary membrane 104 may slide or pull away from the membrane 102 during application to a patient. Such operations may be illustrated in later figures.

The membrane 102 may have an ‘up’ and ‘down’ orientation in some cases. In a typical amniotic membrane 102, there may be stromal surface and an epithelial surface, where the stromal surface may be placed against the patient during surgery.

A typical amniotic membrane 102 may be difficult to determine which surface should be ‘up’ or ‘down’ during surgery. During membrane harvesting and processing, the membrane may be kept in a specific orientation, then a piece of primary backing 104 may be placed on top of the membrane 102. The primary backing 104 may be placed on the surface of the membrane 102 facing away from the patient during surgery.

Because the membrane 102 may be difficult to identify the proper ‘top’ or ‘bottom’ side, the membrane 102 may have a notch or other geometric feature. The feature may be such that a surgeon may be able to verify that the membrane may be in the correct orientation. In a typical geometric feature, a notch may be cut into the upper left hand corner of a rectangular piece of membrane 102, for example. Other geometries may also be used to indicate orientation.

The membrane 102 may be illustrated as a square membrane. However, many different shapes and sizes of membrane 102 may be offered. In some applications, membrane 102 may be round, oval, elliptical, square, rectangular, or any other geometry.

The secondary backing 106 may cover a smaller portion of the membrane 102 than the primary backing 104. In many cases, the secondary backing 106 may have a tab, section, or other area that may extend past the edge of the membrane 102. The tab portion may be used by a surgeon to remove the secondary backing from between the membrane 102 and the patient during the process of applying the membrane during surgery.

The secondary backing 106 may be smaller than the membrane 102 in at least one dimension. The area where the secondary backing 106 may not come in contact with the membrane 102 may expose the membrane for direct contact with a patient. The membrane sandwich may be placed onto a patient such that the unexposed areas of the membrane 102 contact the patient. After removing the primary backing 102, the secondary backing 106 may be slid from between the patient and membrane 102, leaving the membrane 102 properly positioned on the patient.

The secondary backing 106 may be opaque or have some other color or visual identifier. For example, the secondary backing 106 may be white, yellow, blue, or some other color to help identify the secondary backing 106 when in contact with the patient. By having the secondary backing 106 a color that stands out from the patient, a surgeon may be able to quickly verify that the secondary backing 106 has been removed and discarded.

The sandwiched membrane 102 may be stored in an envelope 108. The envelope 108 may contain a liquid preservation media, which may typically be clear in color. The membrane 102 may be immersed in the preservation media while in the envelope 108.

The envelope 108 may be a flat, planar envelope. In many cases, one side may be translucent, such that a surgeon or other personnel may be able to inspect the membrane while still in the packaging. Some packaging may have one side translucent and a second side opaque.

The envelope 108 may be heat sealed, as illustrated with a heat seal 112. Many such packages may be opened by cutting the package along an edge to expose the membrane sandwich immersed in the preservation media.

Some envelopes 108 may have a port 110 through which preservation media may be introduced into the package. Some such ports may be a Luer connection where preservation media may be introduced using a syringe, for example.

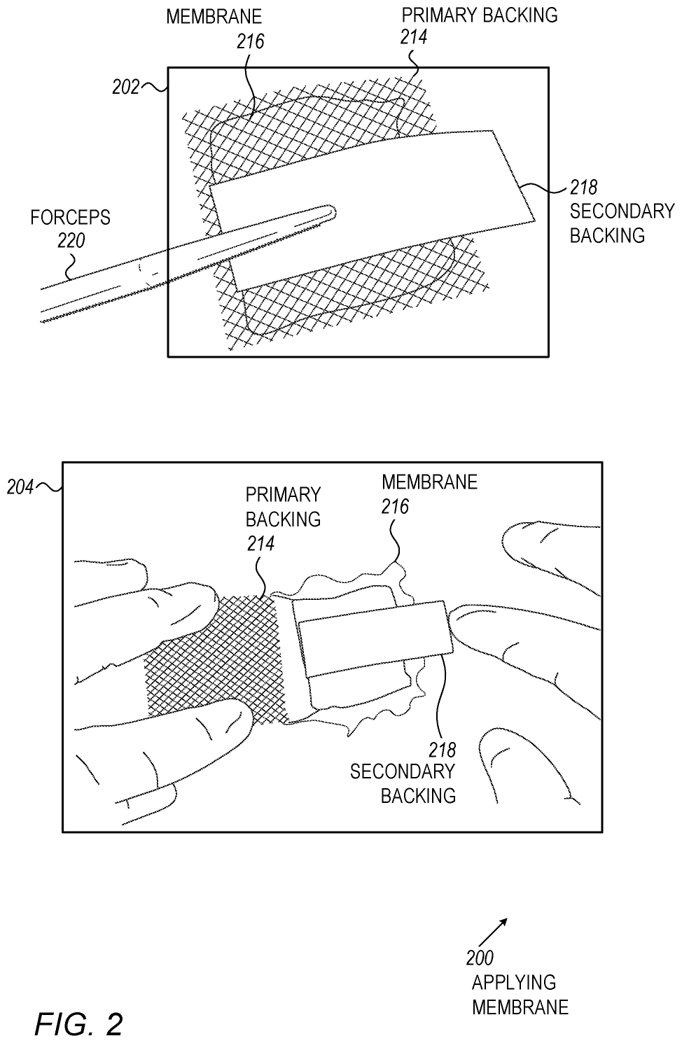

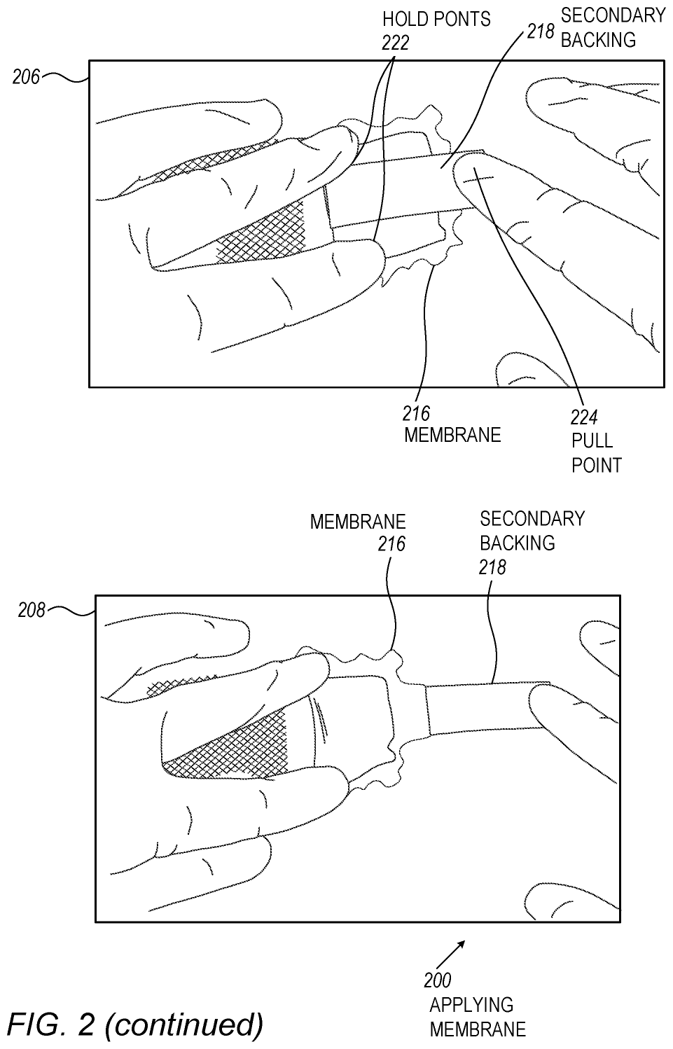

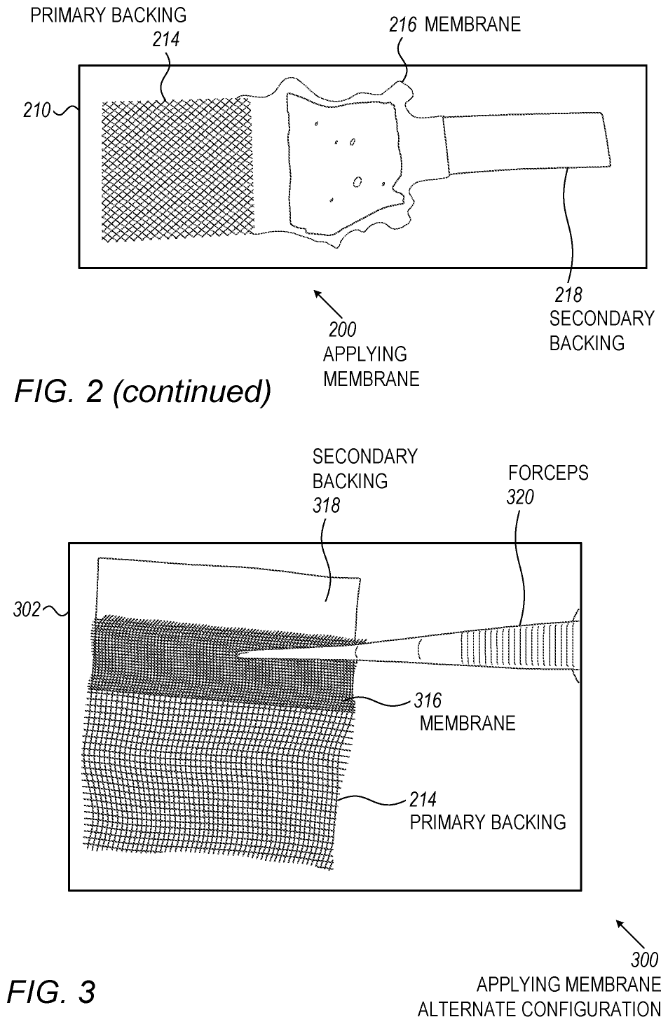

FIG. 2 is an example 200 of a sequence for applying a membrane. In the example 200, the membrane may be illustrated as being applied to a flat surface, which is a substitute for a patient. The sequence 202, 204, 206, 208, and 210 may illustrate the steps for applying the sandwiched membrane.

The sandwich in sequence 202 may have a primary backing 214, the membrane 216, and a secondary backing 218. The sandwich may be manipulated by forceps 220. A surgeon may open a storage and transport envelope to extract the sandwich assembly using forceps 220. The surgeon may inspect the membrane 216 and place the sandwich assembly onto a patient with the primary backing 214 up.

Because the membrane 216 may be spread out on the primary backing 214, the primary backing 214 may assist the surgeon in placing the membrane. The primary backing 214 may be strong enough to keep the membrane 216 from folding over on itself, and the membrane 216 may adhere to the primary backing 214 through surface tension of the wet membrane.

In sequence 204, the membrane 216 may be placed downwards on the patient such that the areas not covered by the secondary backing 218 are in contact with the patient. The primary backing 214 may be placed upwards, away from the patient.

The primary backing 214 may be slid away and off of the membrane 216. In many cases, the wet membrane 216 may adhere to the primary membrane 216 using surface tension during manipulation of the sandwich assembly. At the same time, the fluid between the membrane 216 and the primary backing 214 may allow the primary backing 214 to slide off membrane 216 during the step 204.

In sequence 206, a surgeon may hold the membrane 216 at the hold points 222, while gently removing the secondary backing 218 by pulling the secondary backing 218 at a pull point 224. During this step, the surgeon may press the membrane 216 to the patient to keep the membrane 216 from moving, while at the same time, the secondary backing 218 may be slid out from between the membrane 218 and the patient.

Sequence 208 may show the membrane 216 spread out and in contact with a patient, while the secondary backing 218 may be completely removed from between the patient and the membrane 216.

Sequence 210 may show the primary backing 214, membrane 216, and secondary backing 218 after a successful application.

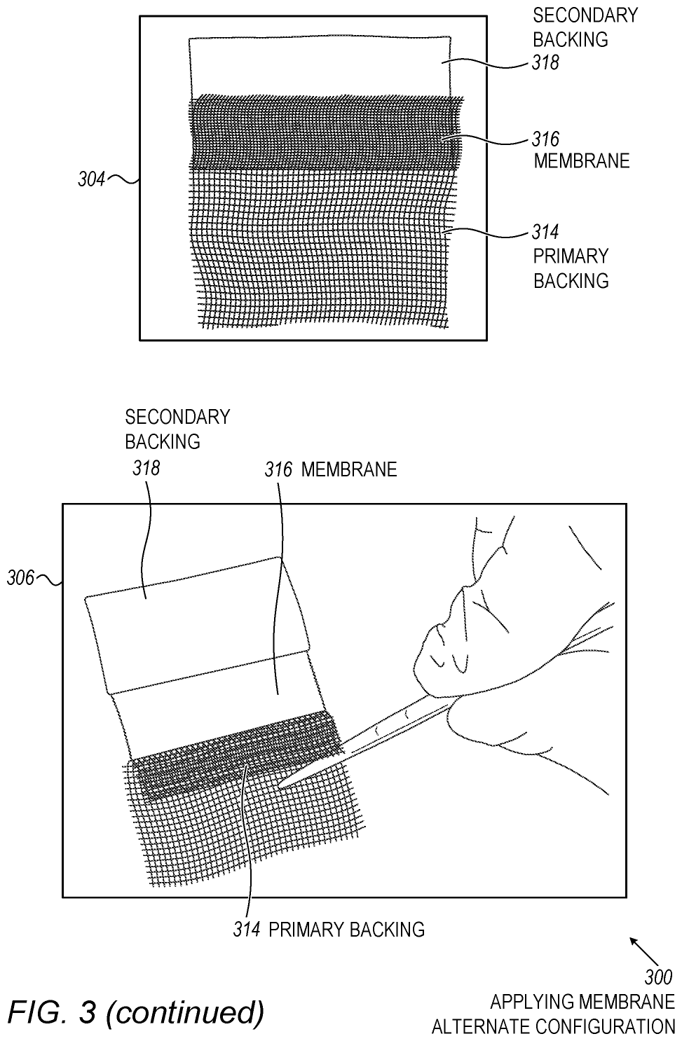

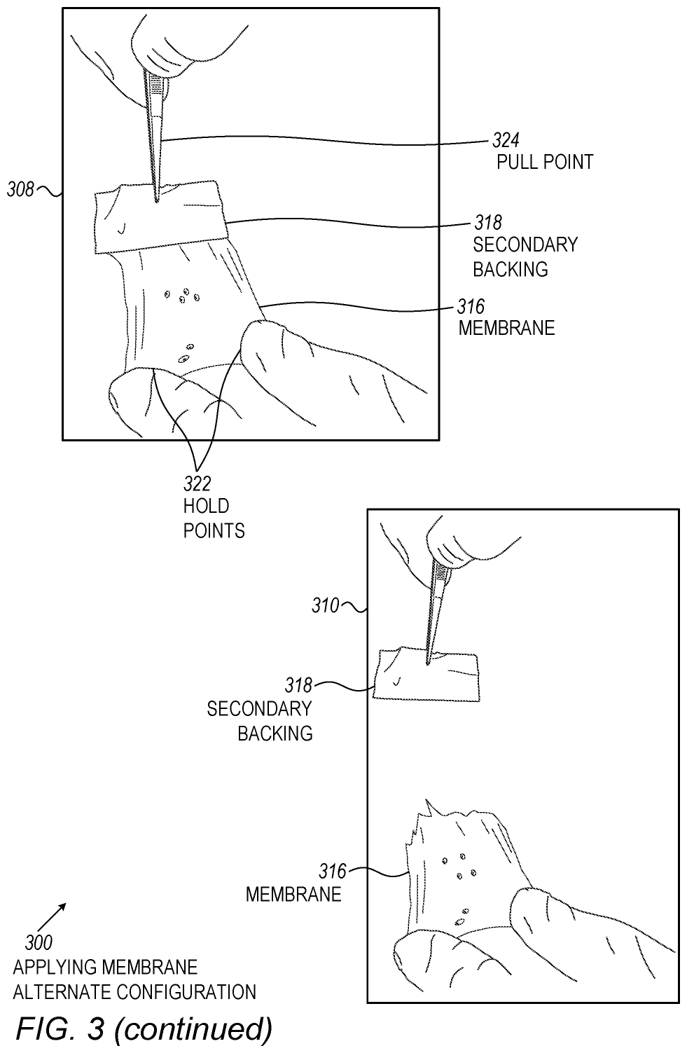

FIG. 3 is an illustration of an example 300 showing a different sequence for installation. Example 300 shows sequences 302, 304, 306, 308, and 310 for applying a membrane.

Example 300 shows a sequence where a primary backing 314 may be peeled away from the membrane 316 as opposed to a sliding action. Further, example 300 shows a sandwich assembly where a secondary backing 318 may be illustrated as a tabbed area that extends past one edge of the membrane 316 on one edge, leaving a larger portion of the membrane exposed to the patient.

Forceps 320 may be used to retrieve the sandwich assembly from a transportation package and preservation media. The sandwich assembly may have a primary backing 314, the membrane 316, and a secondary backing 318.

The primary backing 314 may be about the same size as the membrane 316. In the illustration, the primary backing 314 and membrane 316 may extend upwards (in the illustration) to about halfway up the secondary backing 318. This configuration has about half of the secondary backing 318 in contact with the membrane 314.

In sequence 304, the sandwich assembly may be placed on the patient, with the secondary backing 318 and membrane 316 in contact with the patient, and the primary backing 314 on top, away from the patient.

In sequence 306, the primary backing 314 may be peeled up and away from the membrane 316. The peeling action may begin at the edge where the secondary backing 318 is located. The surgeon may use forceps or a finger to keep the membrane pushed against the patient while peeling away the primary backing 314.

In sequence 308, the surgeon may use fingers to hold the membrane 316 at one, two, or more hold points 322, while gently sliding the secondary backing 318 at a pull point 324.

After pulling away the secondary backing 318 in sequence 310, the membrane 316 may be successfully placed on the patient in a fully spread-out and smooth condition.

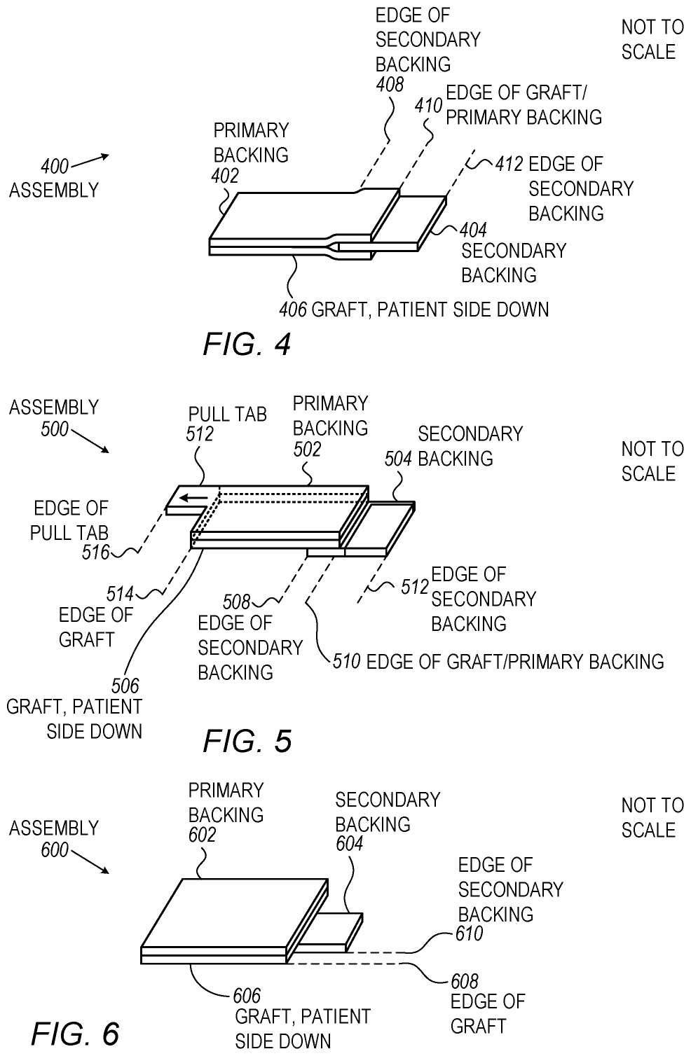

FIG. 4 is a diagram illustration of an embodiment 400 showing a graft assembly with backing. FIG. 4 is not to scale.

A primary backing 402 and secondary backing 404 are shown with a graft 406. The graft 406 may be a membrane tissue that may be used in human surgery, and is shown with the patient-side facing downward.

The assembly 400 may illustrate a use case where a secondary backing 404 may be placed between the primary backing 402 and the graft 406. An edge of the secondary backing 408 may be inside the edge 410 of the primary backing 402 and the graft 406, and the edge 412 of the secondary backing 404 may protrude past the edge 410.

The assembly 400 may illustrate one use case where the full planar surface of the graft 406 may be exposed to a patient. The secondary backing 404 may be held in place using fingers, for example, in the area between the edge 410 and edge 412. While holding down the secondary backing 404, a surgeon may peel away the primary backing 402 by teasing apart the primary backing 402 and secondary backing 404, then peeling away the primary backing 402. Once the primary backing 402 may be removed, the graft 406 may be sutured or held in place by finger pressure, and the secondary backing 404 may be removed.

FIG. 5 is a diagram illustration of an embodiment 500 showing a graft assembly with backing. FIG. 5 is not to scale.

A primary backing 502 and secondary backing 504 are shown with a graft 506. The graft 506 may be a membrane tissue that may be used in human surgery, and is shown with the patient-side facing downward.

The assembly 500 may illustrate a use case where a pull tab 512 connected to the primary backing 502 may assist in removing the primary backing 502 by sliding the primary backing 502 from the graft 506.

The assembly 500 may illustrate a use case where the secondary backing 504 may be placed between the graft 506 and the patient. In this case, the secondary backing 504 may extend over the graft 506 in the area between the edge 508 of the secondary backing and the edge 510 of the graft 504.

The secondary backing 504 may be selected from materials so that the graft 506 may adhere to the secondary backing 504. The adhesion may be achieved by material selection, hydrophobic/hydrophilic material, pore size, surface finish, or other features.

The assembly 500 may be used by placing the graft 506 on the patient in the area to be treated. Once placed, a surgeon may hold the assembly in place using the exposed area of the secondary backing 504 between the edges 510 and 512, then remove the primary backing 502 by pulling on the pull tab 512. Once the primary backing 502 is removed, the surgeon may then hold the graft 506 against the patient, and pull the secondary backing 504 out from under the graft 506.

FIG. 6 is a diagram illustration of an embodiment 600 showing a graft assembly with backing. FIG. 6 is not to scale.

A primary backing 602 and secondary backing 604 are shown with a graft 606. The graft 606 may be a membrane tissue that may be used in human surgery, and is shown with the patient-side facing downward.

Assembly 600 may illustrate a version where the secondary backing 604 may be narrower than the full width of the graft 606. The secondary backing 604 may have an edge 610 that may be inset from the edge 608 of the graft 606.

The use case of assembly 600 may be similar to that of assembly 500, where the primary backing 602 may be slid or peeled away while holding the secondary backing 604 towards the patient.

In the case of assembly 600, the adhesion between the secondary backing 604 and the graft 606 may be provided by the surface area in contact between the two elements. In assembly 500, the full width of the secondary backing 504 may be in contact with the graft 506, while in assembly 600, only a portion of the full width of the secondary backing 604 may be in contact with the graft 606.

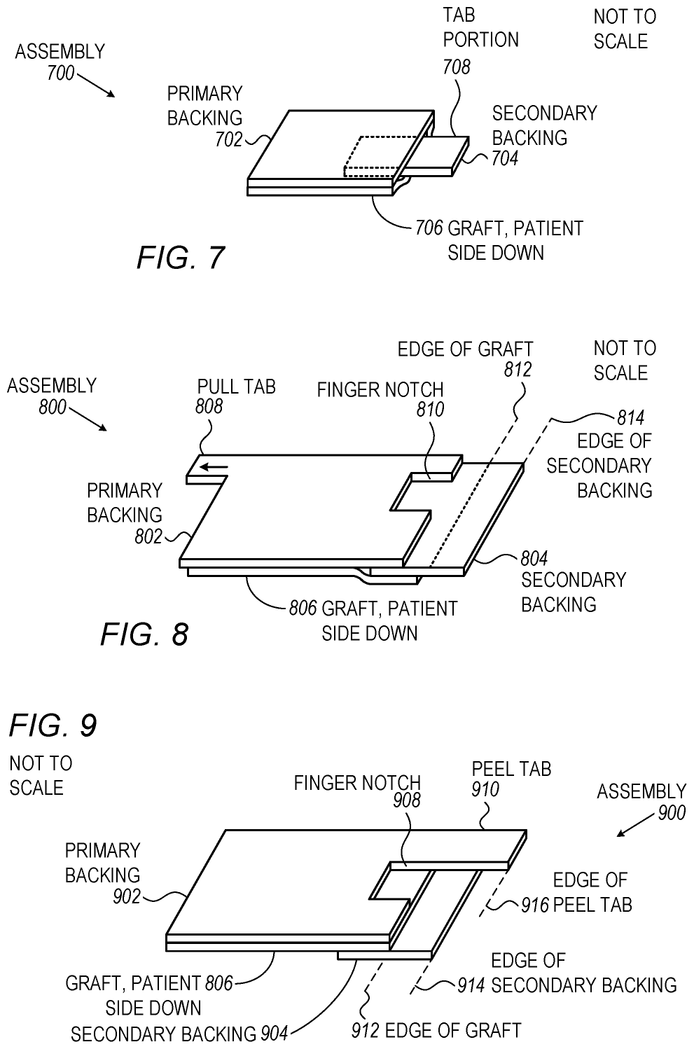

FIG. 7 is a diagram illustration of an embodiment 700 showing a graft assembly with backing. FIG. 7 is not to scale.

A primary backing 702 and secondary backing 704 are shown with a graft 706. The graft 706 may be a membrane tissue that may be used in human surgery, and is shown with the patient-side facing downward.

Assembly 700 may be similar to assembly 600, except the secondary backing 704 may be located between the primary backing 702 and the graft 706. The secondary backing 704 may form a tab portion 708 similar to that of assembly 600.

The assembly 700 may operate in a similar manner to that of assembly 600, however, the operation of the secondary backing 704. In assembly 600, the secondary backing 604 may provide a slight adhesive force between the graft 606 and the secondary backing 604 due to flatwise adhesion or other forces. In assembly 700, the secondary backing 704 may hold the graft 706 in place using more mechanical forces because the secondary backing 704 may be placed over and on top of the graft 706.

FIG. 8 is a diagram illustration of an embodiment 800 showing a graft assembly with backing. FIG. 8 is not to scale.

A primary backing 802 and secondary backing 804 are shown with a graft 806. The graft 806 may be a membrane tissue that may be used in human surgery, and is shown with the patient-side facing downward.

Assembly 800 may be another use case where the secondary backing 804 may be located between the primary backing 802 and the graft 806. In this case, a pull tab 808 and finger notch 810 may be included. Also in this version, the edge 812 of the graft 806 may extend past the closest edge of the primary backing 802.

The assembly 800 may be used by placing the assembly on a patient with the graft 806 in contact with the patient. A surgeon may place one finger in the finger notch 810 to hold the secondary backing 804 and graft 806 in place, while pulling on the pull tab 808 to slide the primary backing 802 off the graft 806. Once the primary backing 802 may be removed, the surgeon may remove the secondary backing 804.

Assembly 800 is one example where the primary backing 802 may have one area that may extend past the planar area of the graft 806 in the form of the pull tab 808. At the same time, the primary backing 802 may be smaller than the graft 806 in at least one dimension, that being the extension of the graft 806 at the edge 812 that extends past the edge of the primary backing 802.

FIG. 9 is a diagram illustration of an embodiment 900 showing a graft assembly with backing. FIG. 9 is not to scale.

A primary backing 902 and secondary backing 904 are shown with a graft 906. The graft 906 may be a membrane tissue that may be used in human surgery, and is shown with the patient-side facing downward.

Assembly 900 is an example of an assembly designed for removing the primary backing 902 by peeling rather than pulling or sliding. The primary backing 902 may have a finger notch 908 as well as a peel tab 910. The secondary backing 904 may be located between the graft 906 and the patient.

To use the assembly 900, the assembly may be placed on a patient with the graft 906 in contact with the patient. A surgeon may hold the graft 906 in place by placing a finger in the finger notch 908. While holding the assembly in place, the surgeon may grasp the peel tab 910 and peel the primary backing upwards. Because the finger in the finger notch 908, the graft 906 may be held in place during the peeling motion and thereby separate from the primary backing 902.

Assembly 900 illustrates another version where the primary backing 902 may extend past both the graft 906 and the secondary backing 904. The edge 912 shows the end of the graft 906, edge 914 shows the end of the secondary backing 904, and the edge 914 shows the end of the peel tab 910.

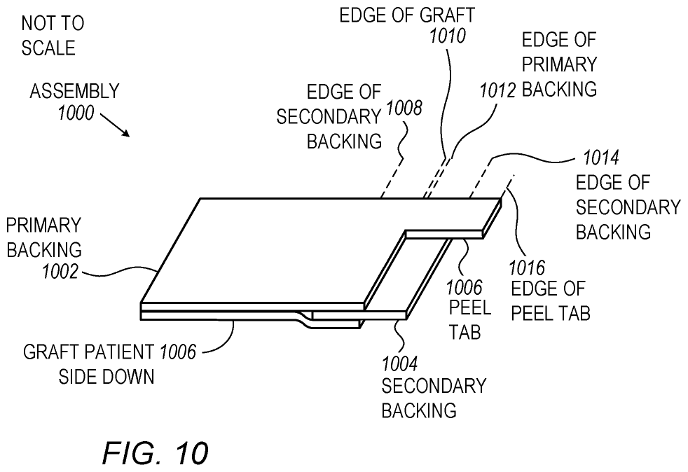

FIG. 10 is a diagram illustration of an embodiment 1000 showing a graft assembly with backing. FIG. 10 is not to scale.

A primary backing 1002 and secondary backing 1004 are shown with a graft 1006. The graft 1006 may be a membrane tissue that may be used in human surgery, and is shown with the patient-side facing downward.

Assembly 1000 may be a version similar to assembly 800, but with a peel tab rather than pull tab. The primary backing 1002 may have a peel tab 1006 that may extend over the secondary backing 1004. In the example of assembly 1000, the edge 1008 of the secondary backing may extend between the primary backing 1002 and graft 1006. The edge 1010 of the graft 1006 may be within the edge 1012 of the primary backing 1002. The edge 1014 of the secondary backing 1004 may extend further still, along with the edge 1016 of the peel tab 1006

The assembly 1000 may be used by placing the assembly on a patient, such that the patient-side of the graft 1006 may be touching the patient. A surgeon may hold the secondary backing 1006 with a finger, while grasping the peel tab 1006 and lifting the primary backing 1002 from the graft 1006. Next, the surgeon may suture or otherwise hold the graft 1006 in place while removing the secondary backing 1004.

The foregoing description of the subject matter has been presented for purposes of illustration and description. It is not intended to be exhaustive or to limit the subject matter to the precise form disclosed, and other modifications and variations may be possible in light of the above teachings. The embodiment was chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments except insofar as limited by the prior art.

1. A membrane product comprising:
a transportation and storage assembly comprising:
a portion of intact membrane tissue, said intact membrane tissue having a first side and a second side, said second side being applied to a patient during treatment, said intact membrane tissue further having a planar area;
a primary backing material supporting said portion of intact membrane tissue on said first side, said primary backing material having a first backing material;
said primary backing material having a surface area at least approximately as large as said planar area;
a secondary backing material supporting said portion of intact membrane, said secondary backing material having a second backing material;
said secondary backing material being smaller than said intact membrane tissue on at least one dimension and having at least a portion of said secondary backing material extending past said intact membrane tissue on at least one dimension;
said first backing material having at least one different physical characteristic than said second backing material.

2. The membrane product of claim 1 further comprising:
a planar envelope comprising at least one translucent layer into which said transportation and storage assembly is sealed.

3. The membrane product of claim 2 further comprising:
a liquid preservation medium in which said transportation and storage assembly is immersed when encapsulated in said planar envelope.

4-5. (canceled)

6. The membrane product of claim 3, said package comprising a syringe port.

7. The membrane product of claim 1, said at least one physical characteristic being that said primary backing material having a first color and said second backing material having a second color.

8-9. (canceled)

10. The membrane product of claim 1, said at least one physical characteristic being that first backing material having a pore size and said second backing material having a second pore size.

11-14. (canceled)

15. The membrane product of claim 1, said at least one physical characteristic being that first backing material having a different hydrophilic property than said second backing material.

16-18. (canceled)

19. The membrane product of claim 1, said second backing material being placed against said second side of said intact membrane tissue.

20. The membrane product of claim 1, said second backing material being placed between said primary backing material and said intact membrane tissue.

21-24. (canceled)

25. The membrane product of claim 1, said at least one physical characteristic being that said first backing material having a first texture and said second backing material having a second texture.

26. (canceled)

27. The membrane product of claim 1, said second backing material being sized to overlay less than 10% of said planar area of said intact membrane tissue.

28. The membrane product of claim 1, said second backing material being sized to overlay less than 25% of said planar area of said intact membrane tissue.

29. The membrane product of claim 1, said second backing material being sized to overlay less than 50% of said planar area of said intact membrane tissue.

30. The membrane product of claim 1, said second backing material being sized to overlay less than 75% of said planar area of said intact membrane tissue.

31. The membrane product of claim 1, said portion of said intact membrane tissue overlying at least 75% of said planar area of said intact membrane tissue.

32. The membrane product of claim 1, said portion of said intact membrane tissue overlying at least 90% of said planar area of said intact membrane tissue.

33-36. (canceled)

37. The membrane product of claim 1, said secondary backing material being placed against said second side of said portion of intact membrane tissue.

38. An amniotic membrane product comprising:
a transportation and storage assembly comprising:
a portion of intact amniotic membrane tissue, said intact amniotic membrane tissue having a first side and a second side, said second side being applied to a patient during treatment, said intact amniotic membrane tissue further having a planar area;
a primary backing material supporting said portion of intact membrane tissue on said first side, said primary backing material having a first backing material;
said primary backing material having a surface area at least approximately as large as said planar area;
a secondary backing material supporting said portion of intact membrane, said secondary backing material having a second backing material;
said secondary backing material being smaller than said intact membrane tissue on at least one dimension and having at least a portion of said secondary backing material extending past said intact membrane tissue on at least one dimension;
said first backing material having at least one different physical characteristic than said second backing material;
a preservation media derived from amniotic fluid.

39-46. (canceled)

47. A method of treatment comprising:
receiving a tissue assembly comprising:
a portion of intact membrane tissue, said intact membrane tissue having a first side and a second side, said second side being applied to a patient during treatment, said intact membrane tissue further having a planar area;
a primary backing material supporting said portion of intact membrane tissue on said first side, said primary backing material having a first backing material;
said primary backing material having a surface area at least approximately as large as said planar area;
a secondary backing material supporting said portion of intact membrane tissue on said second side, said secondary backing material having a second backing material;
said secondary backing material being smaller than said intact membrane tissue on at least one dimension and having at least a portion of said secondary backing material extending past said intact membrane tissue on at least one dimension;
placing said tissue assembly on a treatment area such that at least a first portion of said portion of said intact membrane tissue is against a treatment area;
removing said primary backing material from said portion of intact membrane tissue;
holding said intact membrane tissue to said treatment area and removing said secondary backing material to apply said portion of intact membrane tissue to said treatment area.

48-57. (canceled)
